(19) United States
(12) Patent Application Publication
(10) Pub. No.: US 2026/0294700 A1
(43) Pub. Date: Oct. 1, 2026

(54) PIERCING CLOSURE ASSISTANCE MEMBER, DRUG DELIVERY DEVICE AND METHOD

(71) Applicant: SANOFI, Paris (FR)

(72) Inventors: Claus GEIGER, Frankfurt am Main (DE); Tim GLÄSSER, Rüsselsheim (DE); Moritz KEIM, Frankfurt am Main (DE); Matthias RAU, Rüsselsheim (DE); Michael SCHABBACH, Frankfurt am Main (DE); Marc SCHADER, Frankfurt am Main (DE); Christiane SCHNEIDER, Frankfurt am Main (DE); Michele VOLK, Frankfurt am Main (DE); Stefan WENDLAND, Frankfurt am Main (DE)

(21) Appl. No.: 19/489,888

(22) PCT Filed: Jun. 5, 2024
(86) PCT No.: PCT/EP2024/065438

§ 371 Date: Dec. 3, 2025

(30) Foreign Application Priority Data
Jun. 5, 2023 (EP) 23315229.7

(51) Int. Cl. A61F 15/00

(52) U.S. Cl. CPC A61F 15/005

(57) ABSTRACT

An assistance member for applying a piercing closure element is disclosed. The assistance member includes a holder configured to retain the piercing closure element and to facilitate its application to a piercing site on the skin of a subject. The piercing closure element includes a surface configured for adhesive attachment to the subject's skin. In use, the holder positions and supports the piercing closure element during placement, and the adhesive surface secures the element to the skin to assist closure of the piercing site.

### CROSS-REFERENCE TO RELATED APPLICATIONS

The present application is the national stage entry of International Patent Application No. PCT/EP2024/065438, filed on Jun. 5, 2024, and claims priority to European Application No. EP 23315229.7, filed on Jun. 5, 2023, the disclosures of which are incorporated herein by reference.

### TECHNICAL FIELD

The disclosure relates to a piercing closure assistance member and to a drug delivery device. The drug delivery device may be an autoinjector or a manually or semi-automatically operated device. An energy-storing element may be used in autoinjectors as well as in semi-automatically operated devices in order to deliver the driving force for the injection operation. The energy-storing element may be biased in the factory or by the user prior to use.

The drug may comprise insulin or GLP-1 (Glucagon-Like Peptide). However, other drugs may also be injected.

### BACKGROUND

A band-aid, bandage or plaster or other piercing closure elements may be applied to the skin of a patient after injection of a drug, e.g. to prevent that dust, germs, viruses, bacteria, etc. to come into contact with the wound generated by e.g. the insertion of a needle. Thus, the piercing closure elements mentioned may improve sterility.

The same issues may be relevant for other insertion and/or injection devices, e.g. lancets, syringes, prefilled syringes, etc.

It is an object of the disclosure to provide a member for assisting in the closure of a piercing or piercing opening and/or to provide an improved drug delivery device. The member/device should be preferably easily and/or comfortably to use and/or comprise as few parts as possible. Furthermore, preferably the member/drug delivery device should provide more user comfort compared to known devices. Furthermore, a corresponding piercing closure element and a corresponding method shall be provided.

### SUMMARY

This object is or these objects are solved by the member disclosed herein. According to an embodiment, a piercing closure assistance member includes a member holder that is configured to hold the piercing closure element while the piercing closure element is applied to a piercing site on the skin of a subject.

According to a further embodiment, the skin may comprise a piercing puncture, e.g. a hole that has a depth that may be greater than its width, e.g. twice triple or fourfold. However, the puncture may be in living tissue. Therefore, there may be damage of the tissue within a column that corresponds to the puncture Pu.

According to an embodiment, the piercing closure member may comprise a surface that is configured to be adhesively attached to the skin of the subject. The piercing closure element may be a band-aid, bandage or plaster that may cover the puncture. According to a further embodiment, the piercing closure element, e.g. a band-aid, bandage or plaster may comprise a central hole, e.g. a puncture may not be covered by but surrounded by the piercing closure element, e.g. a band-aid, bandage or plaster. In both cases, the piercing closure element may be stretched or non-stretched in the conformation in which it is applied to the skin and/or may compress the skin after release from the piercing closure assisting member or may not compress the skin.

According to a further aspect, a piercing closure element application device is provided. The piercing closure element application device includes a piercing closure assistance member that is configured to apply a piercing closure element to a skin of a subject.

According to a further aspect, the piercing closure element application device includes a main housing part. According to a further aspect, there may be no drug administration element and/or no drug container and/or no retaining space for such a drug container within the main housing. According to a further aspect, the piercing closure element application device may be used as a lancet or cannula or a needle for sampling a biological probe, e.g. a blood probe from a person/animal. According to a further aspect, the piercing closure element application device may not even comprise a needle, nozzle, cannula or other fluid transportation element. Thus, the wound or puncture may be generated by another device prior to application of the piercing closure element using the piercing closure element application device.

Thus, the device, e.g. the piercing closure element application device may be a device that is used to ease application of a piercing closure element to a wound, e.g. a piercing closure element that is stretched over the wound or that is non-stretched or only marginally stretched. Thus, it may be possible by using the piercing closure element application device to apply the piercing closure element using only one hand.

According to an embodiment, the piercing closure assistance member may be configured to apply the piercing closure element in a stretched conformation. The piercing closure element may be stretched e.g. at least 10 percent or at least 20 percent of the non-stretched length of the piercing closure element in the stretching direction. Stretching may be less than e.g. 100 percent or less than 50 percent of the non-stretched length. The stretching may be elastically. Thus, the piercing closure element may be released to its original length if external forces are removed. However, some degree of plastically stretching may be involved as well.

Moreover, in a further embodiment, the piercing closure assistance member may be configured to apply the piercing closure element in a non-stretched conformation or in a marginally stretched conformation, e.g. with a stretching of less than 5 percent or less than 1 percent compared to the non-stretched length.

According to an embodiment, the piercing closure assistance member may be configured to stretch the skin SK before the adhesive surface of the piercing closure member 8a, 8b contacts the skin SK. Thus, e.g. a circumferential outer edge, or arms or other protrusions on the piercing closure assistance member may be used that are configured to stretch skin if the piercing closure assistance member is pressed against the skin of the subject.

Also, a combination of both embodiments may be used, i.e. stretching of the piercing closure element, e.g. of a band-aid and of the skin prior to application of the stretched piercing closure element. There may be a synergistic effect, e.g. the stretching may be enhanced considerably. After release of the stretched piercing closure element and of the stretched skin, the enhanced stretching may result in an enhanced compression of the skin. Enhanced compression of the skin may result in faster wound healing.

According to an embodiment, the piercing closure assistance member further includes a housing body, and/or a shield that is arranged movably relative to the housing body, and/or at least one holding element arranged on the shield.

According to an embodiment, the holding element may be configured to hold the piercing closure element. According to an embodiment, the at least one holding element may be configured and/or arranged such that the relative movement between the movable shield and the housing body releases the piercing closure element from the at least one holding element.

According to an embodiment, the release of the piercing closure element may occur preferably when the shield moves relative to the housing body from a first state to a second state, e.g. by user action.

According to an embodiment, the piercing closure element may extend from a first holding element of the at least one holding elements over a contact surface of the movable shield to a second holding element of the at least one holding element. This may allow to stretch the piercing closure element when it is fastened to the holding elements.

The shield may be movable relative to the housing or the housing may be movable relative to the shield. According to another embodiment the housing as well as the shield may be movable.

According to an embodiment, the at least one holding element may comprise or may consist of a pivotable element. According to an embodiment, the relative movement of the movable shield and the housing body may pivot the pivotable element from a first state to a second state. According to an embodiment, in the first state of the pivotable element, the piercing closure element may be held by the pivotable element, preferably in a stretched conformation. Thus, the piercing closure element may be hold at its ends, e.g. at its edges or its borders. This may simplify attaching of the piercing closure element to the piercing closure assisting member.

According to an embodiment, in the second state of the pivotable element, the piercing closure element may be released or may be releasable from the pivotable element. Release of the piercing closure element may be enhanced by an adhesive surface of the piercing closure element. The adhesive surface may adhere to the skin of a subject. Thus, if the piercing closure assisting member is removed from the skin, the piercing closure element, e.g. a band-aid may stick to the skin thereby enhancing releasing of the piercing closure element from the pivotable elements.

According to an embodiment, the piercing closure assistance member may comprise at least one holding feature that may be configured to extend through a central opening of the piercing closure element and to hold the piercing closure element on the holding feature. Thus, the piercing closure element may be hold at its central portion or central opening. Secure holding of the piercing closure element on the piercing closure assisting member may be guaranteed even if lateral forces are applied to the piercing closure element.

According to a further embodiment, the holding feature may comprise a retaining feature that extends radially outwardly, e.g. a flange. Thus, the piercing closure element may be hold without involving movable parts on the piercing closure assisting member.

According to a further aspect, a drug delivery device is provided. The drug delivery device may comprise a piercing closure assistance member according to one of the embodiments mentioned above.

According to a further embodiment, the drug delivery device includes a main housing body and/or a retaining space for a drug container within the main housing body.

According to a further embodiment, the drug delivery device may be an autoinjector, e.g. comprising a drive spring and a plunger adapted to forward a force generated by the drive spring to a stopper/plug within a medicament/drug container in order to expel the drug, a pen-injector, e.g. of the dial (axial) extension type, a patch pump, an LVD (large volume device), e.g. more than 5 ml of drug, e.g. within 5 minutes to 2 hours.

According to a further embodiment, the drug delivery device may be an autoinjector, especially an FLAI (fast and/or large auto-injector). Large may mean that the injection amount may be in the range of 2 ml (milliliter) to 10 ml or in the range of 2 ml to 5 ml or in the range of 2 ml to 3 ml. Fast may mean that even a large amount of medicament is injected within a range of 5 s (second) to 20 s, e.g. less than 20 s for 3 ml or for a volume in the range of 2 ml to 3 ml or e.g. less than 30 s for 5 ml or for a volume in the range of 3 ml to 5 ml. The viscosity of the medicament may be in the range of 1 cp to 50 cp (centipoise, gram/(centimeter*second)), e.g. in the range of 3 cp to 50 cp or in the range of 15 cp to 30 cp. The drug administration element, e.g. a needle may have a diameter ranging from about 25 Gauge to about 31 Gauge in size. Common sizes are 27 and 29 Gauge. However, sizes of 25 Gauge (outer diameter 0.5 mm) may be used for fast and large autoinjectors. Larger volumes of medicaments may be used e.g. for medicaments against auto-immune diseases, especially medicaments comprising antibodies, e.g. monoclonal antibodies.

However, the concepts described in this document may also be applied to other drug injection devices, e.g. to auto-injectors injecting volumes in the range or 1 ml to 2.5 ml, e.g. in less than 15 seconds or in less than 20 seconds.

According to a further embodiment, the housing may comprise an outer surface that is not surrounded by further parts of the drug delivery device. The outer surface may be a gripping surface that is gripped by a user or patient during usage of the drug delivery device, e.g. during injection of a drug into a body of a patient or of an animal.

According to a further embodiment the drug delivery device has a longitudinal axis. The housing may extend along the longitudinal axis. The housing may comprise a proximal end and a distal end. The distal end may be closer to a needle of the drug delivery device than the proximal end. The housing may comprise only one part or several parts, e.g. a main part, e.g. an elongated cylindrical body and preferably a rear housing part that closes the main part of the housing from its rear side, e.g. at the proximal end of the housing.

According to a further embodiment, a drug administration element may be fluidically coupled or may be fluidically coupleable to the drug reservoir. The drug administration element may define or may form a drug outlet of the drug delivery device. A typical example of a drug administration element is a needle. However, nozzles or other drug administration members may be used as well. The drug reservoir may be a container, e.g. a cartridge with a distal septum that may be penetrated by a needle, a syringe, a plastic bag or other fluid storage part.

According to an embodiment, the drug delivery device includes a main housing body, and/or a retaining space for a drug container defined within the main housing body, and/or a movable shield that may be axially movable relative to the main housing body.

According to an embodiment, the movable shield may be configured to cover the drug administration member at least in a first state and to expose the drug administration member in a second state.

According to an embodiment, the movable shield includes the piercing closure assistance member.

According to an embodiment, the piercing closure assistance member includes at least one holding element that may be configured to hold the piercing closure element.

According to an embodiment, the at least one holding element may be configured and/or may be arranged such that the relative movement between the movable shield and the main housing body may release or may help to release the piercing closure element from the at least one holding element, preferably when the movable shield moves from the first state to the second state.

According to an embodiment, the piercing closure element may extend from a first holding element of the at least one holding elements over a contact surface of the movable shield to a second holding element of the at least one holding element.

According to a further embodiment, the contact surface may be configured to contact the skin of a patient and/or the piercing closure element, preferably a portion of the piercing closure element that is arranged between the skin and the contact surface.

According to a further embodiment, the contact surface defines an aperture through which a drug administration element, e.g. a needle, may extend, e.g. in second state. According to a further embodiment, the piercing closure element may extend over an aperture within the movable shield, e.g. over a central aperture, especially within a movable needle shield. The piercing closure element may have a hole where the drug administration member, e.g. a needle may pass through. According to a further embodiment, the piercing closure element may not have a hole for the needle but the needle may have to pierce the piercing closure element in order to insert a drug administration member, e.g. a needle.

According to a further embodiment, the piercing closure element may have the shape of an elongated stripe. This may allow complete or almost complete usage of piercing closure element material, e.g. when using a die cutter or a punch out machine.

According to an embodiment, the at least one holding element may comprise or may consist of a pivotable element. According to an embodiment, the relative movement of the movable shield and of the main housing body may pivot the pivotable element from a first state to a second state, e.g. without further user actions.

According to an embodiment, in the first state of the pivotable element, the piercing closure element may be held by the pivotable element, preferably in a stretched conformation.

According to an embodiment, in the second state of the pivotable element, the piercing closure element may be released or may be releasable from the pivotable element. Pivotable elements, e.g. levers, may be produced and/or assembled in a cost-efficient way.

According to a further embodiment, springs, e.g. compression springs, torsion springs or tension spring may be used to bias the pivotable element in the first state and/or in the second state. According to a further embodiment, clamping elements, e.g. protrusions may be used to hold the pivotable element in the first state and/or in the second state.

According to a further embodiment, resilient beams may be used to hold the ends of the piercing closure element.

According to a further embodiment, the relative movement of the movable shield and of the main housing body may push the piercing closure element over the free ends of the pivotable elements or of the resilient elements thereby releasing the piercing closure element from the pivotable elements and form the drug delivery device.

According to an embodiment, the at least one holding element may comprise or consists of a protrusion that extends preferably radially outwards from the movable shield. According to an embodiment, the protrusion may be configured such that relative movement of the movable shield and of the main housing body may push the piercing closure element over the protrusion thereby releasing the piercing closure element from the protrusion. Protrusions are simple mechanical elements. Protrusions are static elements, i.e. elements that do not involve movement. Thus, safe operation may be provided.

According to a further embodiment, the protrusion may be inclined, e.g. in the proximal direction. The protrusion my comprise or may consist of a ramp. The ramp may have e.g. a greater height at a distal end compared to a height on a proximal end of the ramp. However, round protrusion or other shaped protrusions may be used as well.

According to an embodiment, the piercing closure assistance member includes at least one holding feature that is configured to extend through a central opening of the piercing closure element and to hold the piercing closure element on the holding feature. According to an embodiment, the holding feature may comprise a retaining feature that extends radially outwardly. According to a further embodiment, the retaining feature may be a flange portion or a circumferential flange. Thus, the piercing closure assistance member may be simple and/or without mechanically movable parts, especially without separate movable parts on a movable shield, e.g. on a movable needle shield.

The piercing closure element may be a circular ring or an oval ring, e.g. there may be a central hole within the piercing closure element. Thus, the piercing closure element may have a geometrical shape and/or contour that allows easy assembling to the piercing closure assistance member, e.g. by automatic assembling using a special machine.

According to a further embodiment, the piercing closure assistance member may be assembled in a factory.

According to a further embodiment, the retaining feature may comprise a round flange, a circular flange or an elliptical flange. According to a further embodiment, several flange portions may be arranged on a circle or on an ellipse.

According to a further embodiment, there may be a cylindrical portion, an elliptical portion or other constant diameter portion or other constant maximal width portion, e.g. proximally adjacent to the flange or to the flange portions, e.g. in order to hold the flange. The outer diameter or outer maximal width of the cylindrical portion, etc. may be adapted to the inner diameter/width of a central hole in the piercing closure element. The relevant geometrical feature of the cylindrical portion may be e.g. only some percent smaller than or greater than the inner diameter of the hole within piercing closure element, e.g. in the range of 1 percent to 5 percent. Equal diameters are possible as well.

According to a further embodiment, the piercing closure element may be pre-stretched on the cylindrical portion.

According to a further embodiment, a chamfered or ramped portion may be used to stretch the piercing closure element or to stretch the piercing closure element further when the drug delivery device or other device is used and/or when the piercing closure element is applied to the skin of a patient.

According to a further embodiment, the flange or the flange portions may have an outer diameter and/or maximal width that may be greater than the inner diameter or maximal inner width of the hole of the piercing closure element, e.g. in the range of 1 percent to 5 percent. Thus, the piercing closure element may be prevented to fall off from the piercing closure assistance member in the initial state of the device, e.g. of the drug delivery device or other device used to apply the piercing closure element.

According to a further embodiment, the drug delivery device that carries e.g. the round piercing closure element further includes a main housing body, and/or a retaining space for a drug container defined within the main housing body.

According to a further embodiment, the piercing closure assistance member includes a contact surface that extends essentially in a radial direction, e.g. that is arranged perpendicular to a longitudinal axis of the drug delivery device. Thus, the surface normal of the contact surface may point into the distal direction of the drug delivery device.

According to a further embodiment, there may be a distance between the piercing closure element and the contact surface prior to use of the drug delivery device but with the piercing closure element already arranged on the piercing closure assistance member. The distance may be due to a chamfered or ramped portion on the piercing closure assistance member that may be arranged closer to the contact surface than the flange or the flange portions, e.g. more proximally compared to the position of the flange or the flange portions

According to a further embodiment, the contact surface may be configured to contact the skin and/or the piercing closure element. According to a further embodiment, the contact surface may comprise an aperture through which the drug administration element, e.g. a needle may extend, e.g. in second state. According to a further embodiment, the piercing closure element may extend around aperture. According to a further embodiment, the aperture may have a diameter/width that may allow a cap of the drug delivery device to pass through the aperture.

As already mentioned above, according to a further embodiment, the piercing closure element may have the shape of a ring, e.g. of a circular ring or of an elliptical ring, preferably a flat ring. This shape may be easily to be produced, e.g. with a die cutting tool or with a punch out machine.

According to an embodiment, the drug delivery device may comprise a movable shield that may be configured to cover the drug administration member at least in a first state and to expose the drug administration member in a second state. According to an embodiment, the movable shield may comprise the at least one holding feature that is configured to hold the piercing closure element, e.g. the round piercing closure element. According to an embodiment the piercing closure assistance member may be integral part of the movable shield.

Thus, the piercing closure assistance member may be formed on a distal part of the movable shield. According to a further embodiment, the piercing closure assistance member may be a part of the drug delivery device that is connected permanently to the drug delivery device.

According to an embodiment, the piercing closure assistance member may be connectable to a distal end of the drug delivery device after assembling of the drug delivery device, preferably in order to attach the drug administration element to the drug container. According to an embodiment, preferably, the drug delivery device may comprise a first connection feature that is configured to be connected to a second connection feature on the piercing closure assistance member.

According to a further embodiment, the connection may be established with only a few hand movements, especially without using a tool. A screw thread, a snap connection, e.g. releasable snap connection, a bayonet connection or other appropriate connection may be used.

According to a further embodiment, the piercing closure assistance member may be formed on a distal part of a connection element that comprises the second connection feature. According to a further embodiment, the piercing closure assistance member may not be mounted on a movable shield but may be arranged static relative to a main housing part, e.g. to the main housing part of the drug delivery device or of a device that is used to apply the piercing closure element without injecting a drug.

According to an embodiment, the piercing closure assistance member may comprise at least one stretching element, preferably a circumferential chamfer or circumferential ramp or at least one chamfer portion or at least one ramped portion. According to an embodiment, the stretching element may be configured to stretch the piercing closure element when the piercing closure element is pushed against the skin of a patient and at the same time against the stretching element. Thus, the stretching element has a simple structure and does not involve separate movable parts and/or separate user action(s).

According to a further embodiment, the piercing closure assistance member may comprise the following sequence form the proximal end to the distal end of the piercing closure assistance member:
A contact surface, e.g. a contact surface that extends essentially radially outwards,
A chamfer or ramp portion, e.g. a stretching portion that has an outer maximum width or outer maximum diameter that is smaller than the outer diameter or maximal outer width of the contact surface,
A constant diameter or maximal width middle portion, e.g. having the same outer diameter or the same maximal outer width as at the beginning of the stretching portion, and
A retaining portion having a larger outer dimeter/outer maximal width than the middle portion, e.g. a circumferential flange or at least one flange portions.

According to a further embodiment, the outer dimeter/outer maximal width of the retaining portion may be less than the outer maximal dimeter/outer maximal width of the chamfer or ramp portion, e.g. in order to ease removal of a ring shaped piercing closure element from the piercing closure assistance member.

According to an aspect, a method is provided for preparing a piercing closure assistance member, comprising applying a piercing closure element to the piercing closure assistance member according to any one of the embodiments mentioned above or to the piercing closure assistance member of a drug delivery device according to one of the embodiments mentioned above.

According to a further embodiment, the method includes fastening at least one end of the piercing closure element preferably manually before applying the piercing closure element to the skin of a patient and/or before injecting a drug into the body of the patient by using the drug delivery device, and/or, optionally, by putting a lateral aperture of the piercing closure element over a holding feature of the drug delivery device or of another piercing closure element application device.

According to a further embodiment, the method includes attaching the piercing closure element preferably automatically to the drug delivery device or to another piercing closure element application device by arranging a central opening of the piercing closure element over a piercing closure assisting member or band-aid application feature of the drug delivery device or of another device.

According to a further embodiment, the method further includes at least one, at least two, or at least three or all of the following features:
a) Optional removal of a cap of the drug delivery device, preferably before fastening a stripe like piercing closure element or after arranging/attaching a ring like piercing closure element at the drug delivery device, and/or
b) Preferably, after fastening/attaching of the piercing closure element to the drug delivery device, optional removal of a cover layer of an adhesive layer of the piercing closure element, and/or
c) After fastening/attaching of the piercing closure element and/or after optional removal of a cover layer, applying the piercing closure element to the skin of a patient by using the drug delivery device to inject a drug into the body of the patient, and/or
d) After any one of the previous features, injection of a drug into the body of the patient, and/or
e) Removal of the drug delivery device from the patient, whereby the piercing closure element is left on the skin of the patient, and/or
f) The piercing closure element pulls together or contracts around a puncture or other wound made by insertion of a needle or other sharp medical device or by the injection of the drug and/or the piercing closure element may cover the puncture.

Step f may prevent bleeding and/or may improve wound healing, especially when using drugs that are injected daily or several times a day, e.g. insulin.

Thus, the same technical effects mentioned above for the piercing closure assisting member or for the drug delivery device may apply to the method.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this description it means or it may mean the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, e.g. for lancets, syringes, prefilled syringes or other injectors.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE DRAWINGS

For a more complete understanding of the presently disclosed concepts and the advantages thereof, reference is now made to the following description in conjunction with the accompanying drawings. The drawings are not drawn to scale. In the drawings the following is shown in:

FIGS. 1A to 1D a cross section of a drug delivery device according to a first embodiment and in different operating states,

FIG. 2 depicts an auto-injector according to a second embodiment,

FIG. 3 depicts the auto-injector according to the second embodiment comprising a piercing closure element stripe arranged in front of a distal opening of a movable needle shield,

FIG. 4 depicts the auto-injector according to the second embodiment in a state in which the needle is inserted and drug delivery may commence,

FIG. 5 depicts the skin of a patient after injection of the drug and with the piercing closure element stripe in a contracted conformation,

FIG. 6 depicts the piercing closure element stripe used in the second embodiment in a top view,

FIG. 7 depicts an auto-injector according to a third embodiment comprising a ring shaped piercing closure element arranged around a distal opening of a movable needle shield,

FIG. 8 depicts the auto-injector according to the third embodiment in a state in which the needle is inserted and drug delivery may commence,

FIG. 9 depicts the auto-injector according to the third embodiment after removal from the skin of a patient,

FIG. 10 depicts the ring shaped piercing closure element used in the second embodiment in a top view, and

FIG. 11 depicts an expanded structural formula, molecular formula, and molecular weight of fitusiran.

### DETAILED DESCRIPTION

Reference may be made to a cylindrical coordinate system, i.e. each position may be defined by three coordinates: axial value (height, distance to zero plane), radial distance to axis and angle between current radial position and a plane that is defined as having angle zero. In this document the words “in an axial position” may mean having an axial coordinate.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

The distal direction may be the direction in which a tip of the needle is oriented to. A proximal direction may be a direction opposite the distal direction. The distal and proximal directions may be aligned along a main axis. The main axis may be colinear with the needle or run through the needle. Any or all moving components of the injection device may be configured to move along the main axis and/or rotate around the main axis if not described otherwise. A radial direction may be any direction orthogonal to the main axis. The main axis may be an axis along which most of the components of the injection device are arranged and/or oriented. The main axis may be an axis of lowest moment of inertia of the injection device or at least close to such an axis.

Reference signs having the same last two digits may refer to same or similar elements if not mentioned otherwise, e.g. main housing part 102 and 302.

Embodiments may relate to drug delivery devices comprising other activation mechanism or operated by a manual driving force. Reference is made e.g. to WO 2014/033195 A1 or to WO 2014/033197 A1 in this regard which are included by reference for all legal purposes. The injection button may provide at least one user interface member for initiating and/or performing a dose delivery operation of the drug delivery device. The (dial) grip or knob may provide a user interface member for initiating and/or performing a dose setting operation using a dose setting surface, e.g. the circumferential surface of the (dial) grip or knob. A delivery surface may be used to initiate dose delivery. The delivery surface may be the proximal P surface of the (dial) grip or knob.

The device may be of the dial extension type, i.e. its length may increase during dose setting or dose dialing. Other injection devices with the same kinematical characteristic of the dial extension and button during dose setting and dose expelling operational mode are known as, for example, Kwikpen® and Savvio® device marketed by Eli Lilly as well as FlexPen®, FlexTouch® and Novopen® 4 device marketed by Novo Nordisk or devices of other manufacturers. An application of the general principles disclosed herein to these devices therefore appears straightforward and further explanations will be omitted. Alternatively, the proposed concepts may be used in devices that are not of the dial extension type but include for instance a torsion spring that may be biased by rotation of a dial knob. Moreover, fully mechanically driven or electromechanically driven drug delivery devices may be used, e.g. comprising an electrical motor.

However, the general principles of the present disclosure are not limited to that kinematical characteristic. Certain other embodiments may be conceived, e.g. for application to other injection devices of Sanofi or devices of other manufacturers where there is a container for a drug that may be moved axially relative to a housing of the drug delivery device in order to insert and/or to retract a needle.

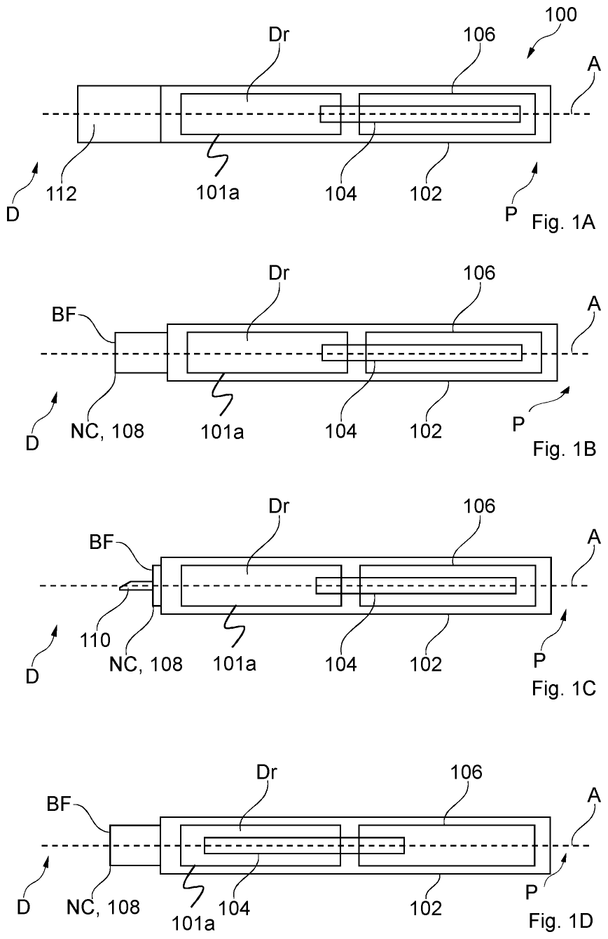

FIGS. 1A through 1D illustrate an embodiment of a drug delivery device 100. Device 100 may be suitable as the device in the drug delivery arrangements described further above and below. The figures show device 100 in different states during its operation.

FIG. 1A illustrates drug delivery device 100 in an initial or as delivered state. Drug delivery device 100 may comprise a housing 102. Housing 102 may be provided to retain and/or may retain a medicament/drug reservoir 101a in its interior. Medicament, e.g. liquid medicament or drug Dr, may be arranged in drug reservoir 101a. Housing 102 may be configured or may be provided to retain and/or may retain a needle 110, see FIG. 1C. The needle 110 may be named as a drug administration element in the claims. However, other drug administration elements are possible as well, e.g. a nozzle. In other words, needle 110 may be arranged or may be arrangeable in housing 102. Needle 110 may be an integral part of drug reservoir 101a, e.g. (permanently or releasable) connected to a body of drug reservoir 101a, or separate from drug reservoir 101a. In the first case, the drug reservoir 101a may be a syringe, e.g. a PFS (Pre Filled Syringe). In the second case, the drug reservoir 101a may be a cartridge. In case a cartridge is used as drug reservoir 101a, initially, drug reservoir 101a and needle 110 can be fluidly disconnected and fluid communication between the interior of drug reservoir 101a and needle 110 may only be established during operation of drug delivery device 100.

A drive mechanism 106 may be provided to drive a drug delivery operation and may expediently be provided in housing 102. Drive mechanism 106 may comprise a plunger rod 104. Drug delivery device 100 may further comprise a drive energy source, e.g. a drive spring, such as a compression spring, (not explicitly shown) or gas or compressed air. The drive energy source may be arranged to drive plunger rod 104 in a distal direction D relative to drug reservoir 101a during the drug delivery operation. During this movement, a stopper or plug, which may be movably retained in drug reservoir 101a and which may seal the medicament container proximally, may be displaced towards an outlet of drug reservoir 101a to dispense drug Dr or medicament retained within the drug reservoir 101a through the outlet. The outlet may be formed or defined by needle 110, see FIG. 1C.

Other potential drive energy sources different from a spring comprise an electrical power cell or battery for driving plunger rod 104 by a motor or a reservoir suitable to provide gas pressure, where the gas pressure can be used to drive the drug delivery operation.

Drug delivery device 100 may be an autoinjector. The energy for driving the drug delivery operation in an autoinjector may be provided by components integral to drug delivery device 100 and does not have to be loaded into the device by the user during the operation of device 100 as is the case in many spring driven pen-type variable dose injectors, where, usually, the energy is loaded into the spring by the user during a dose setting procedure.

Drug delivery device 100 may expediently be a single shot device, i.e. it is provided to dispense only one dose. Drug delivery device 100 may be a disposable drug delivery device 100, that is to say a device 100 which is disposed of after its use. Device 100 may be a pen-type device. A drug reservoir 101a and/or needle 110 may be axially secured within drug delivery device 100, e.g. within housing 102, or may be movable relative to housing 102, e.g. for piercing the skin. In the first case, the user may have to perform the movement for piercing the skin with needle 110. In the second case, piercing of the skin by needle 110 may be driven by a needle insertion mechanism of drug delivery device 100. Automatic needle retraction may be used as well. Drug reservoir 101a may be hold or retained within housing 102 by an optional retaining/supporting member 101b, e.g. by a syringe carrier or other carrier for a drug reservoir 101a. Other embodiments do not comprise an additional syringe carrier or other drug reservoir carrier in addition to the syringe and to housing 102. Thus, drug reservoir 101a may be carried directly by housing 102, e.g. as described below in more detail.

As depicted in FIG. 1A, drug delivery device 100 may further comprises a cap 112. Cap 112 may be arranged at the distal end D of drug delivery device 100. Cap 112 may be detachably connected to the remainder of device 100, e.g. to housing 102 and/or to another component or member of drug delivery device 100. Cap 112 may cover a distal end D of the remainder of drug delivery device 100 and/or a needle passage opening through which needle 110, e.g. the distal needle tip, may pass to pierce the skin from the interior of drug delivery device 100 during or for the drug delivery operation. Cap 112 may comprise a needle cover/shield remover or grabber, e.g. made of metal, which may engage a rigid needle shield/cover RNS, which may cover needle 110 such that the rigid needle shield RNS is removed from needle 100 together with cap 112, e.g. when cap 112 is detached or disconnected from device 100. Housing 102 may expediently cover the majority of the length of drug delivery device 100, e.g. 60 percent or 70 percent or more percent of the entire length of the drug delivery device 100 (with the cap 12 attached and/or with the cap detached).

FIG. 1B illustrates drug delivery device 100 with cap 112 being removed. According to FIG. 1B, device 100 may be in a state ready to be operated, e.g. ready to perform a drug delivery operation when the operation is triggered. As depicted, drug delivery device 100 may further comprises a needle cover NC. Needle cover NC may protrude distally from housing 102 and/or may have been covered by cap 112 when cap 112 was still attached to housing 102. Needle cover NC may be axially movable relative to housing 102 from an initial position or first position to a second position or trigger position. Needle cover NC may be provided to extend beyond the distal tip of needle 110 which may protrude from the housing 102 before the drug delivery operation is commenced, e.g. in a first position. Needle cover NC may be movable in the proximal direction relative to housing 102. During this movement, e.g. before needle cover NC reaches the second position, needle 110 may pierce the skin of the user.

Needle cover NC may serve as a trigger member 108 of the drug delivery device. Needle cover NC as trigger member 108, when displaced proximally from the initial position or first position depicted in FIG. 1B to the second position or trigger position (see FIG. 1C), may automatically initialize the drug delivery operation of drug Dr, preferably when it is in the second position. The drug delivery operation can be initialized by removing a mechanical lock which prevents movement of plunger rod 104 in the distal direction or by moving plunger rod 104 to disengage a mechanical lock using moving needle cover NC. Alternatively, needle cover NC when moved from the first position to the second position and expediently when in the second position may only enable triggering of the drug delivery operation. In this case, a separate trigger member, e.g. a trigger button on the proximal end of the housing 102, may be provided to initiate the drug delivery operation. Operating the trigger button to initiate the drug delivery operation of drug Dr may only be possible when needle cover NC is in the second position. In yet another alternative, needle cover NC may only be provided to prevent needle stick injuries before and/or after use of drug delivery device 100. In this case, needle cover NC may be completely decoupled from drive mechanism 106 and/or may not be involved in triggering or enabling triggering of the drug delivery operation at all.

Needle cover NC may be provided to bear against the skin of a user during injection. Hence, the distal surface of needle cover NC may provide a bearing surface or bearing face BF. Bearing surface BF may delimit and/or extend around a needle passage opening provided in needle cover NC. The bearing surface 16 may be ring-like, oval, elliptic, rectangular, quadratic, etc., circumferentially closed and/or be defined by an inward protrusion protruding radially inwards from an inner wall of needle cover NC, e.g. a distal cylindrical portion thereof. Bearing surface BF may be expediently the distal end surface of needle cover NC, e.g. facing distally.

FIG. 1C illustrates needle cover NC in the second position relative to housing 102. This is the position when the drug delivery operation has been initiated, can be initiated, and/or when the needle pierces the skin, for example. Needle 110 may protrude axially from bearing surface BF of drug delivery device 100 (particularly through the needle passage opening in needle cover NC) and, by the distance with which it protrudes over the bearing surface BF, penetrate the skin (the skin is not shown in this representation, see e.g. skin SK in FIGS. 4, 5, 8 and 9). This distance may be characteristic for or be equal to the injection depth. The device 100 may be maintained in contact with the skin SK until the drug delivery operation of drug Dr has been completed, which may be indicated by an optional audible, tactile, and/or visual indication provided by the drug delivery device 100. Optionally, also the beginning of drug delivery may be signaled to the user.

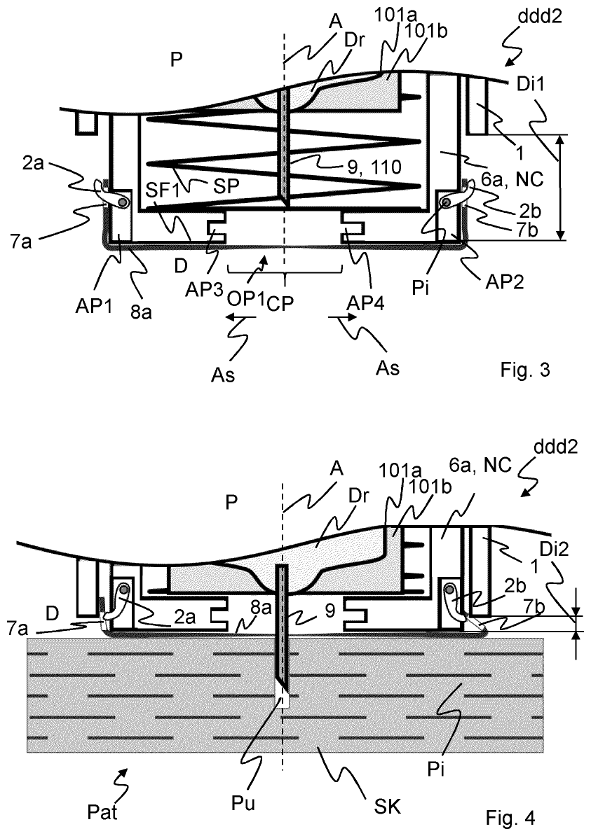

After the drug delivery operation of drug Dr has been completed, e.g. piston rod 104 has moved distally, device 100 may be removed from the skin (see FIG. 1D). Needle cover NC may be biased relative to housing 102 towards the first position by a spring (not shown, see e.g. spring SP in FIGS. 3 and 8). Thus, when device 100 is removed from the skin, needle cover NC may be moved towards the first position with respect to the housing 102. Needle cover NC may be moved distally, e.g. beyond its first position, into a final, third or locked position relative to housing 102. In this position, needle cover NC may expediently be locked axially relative to the housing 102 against movement in the proximal direction P, e.g. by a locking engagement between a locking feature of needle cover NC and housing 102. As it is axially locked, needle cover NC may no longer be displaced proximally relative to housing 102 into the second position and/or into the first position. This may protect the user from needle stick injuries after use. In this state, device 100 may be locked, see FIG. 1D.

Alternatively, there may be no movable needle cover. Thus, a distal device portion may be pressed against the skin of a user. Needle injection may be performed by a needle insertion mechanism. An optional needle retraction mechanism may also be used. Triggering may be performed using a proximal trigger element, e.g. a proximal knob of modified device 100.

Drug delivery device 100 may comprise an electronic unit that may be mechanically connected to a proximal end region P or to another region of drug delivery device 100. The electronic unit may be used not only for drug delivery device 100 but also for other drug delivery devices that are similar or identical to drug delivery device 100. Alternatively, the electronic unit may be an integrated part of the drug delivery device 100. The electronic unit may be used to monitor drug delivery, e.g. amount of dose, time and date.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C. (degrees centigrade, Celsius)), or refrigerated temperatures (e.g., from about −4° C. to about 4° C.). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### Fitusiran as the API for the Medicament in the Device

Fitusiran is a synthetic, chemically modified double-stranded small interfering RNA (siRNA) oligonucleotide covalently linked to a tri-antennary N-acetyl-galactosamine (GaINAc) ligand targeting AT3 mRNA in the liver, thereby suppressing the synthesis of antithrombin. See, e.g., Pasi et al., N Engl J Med. (2017) 377(9):819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GaINAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-
Um-Uf-Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-
Gm-Gm-Uf-Gm-Uf-Um-Af-Am-Cf-Cm-ps-Am-ps-Gm 3′,

wherein
Af=2′-deoxy-2′-fluoroadenosine
Cf=2′-deoxy-2′-fluorocytidine
Gf=2′-deoxy-2′-fluoroguanosine
Uf=2′-deoxy-2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

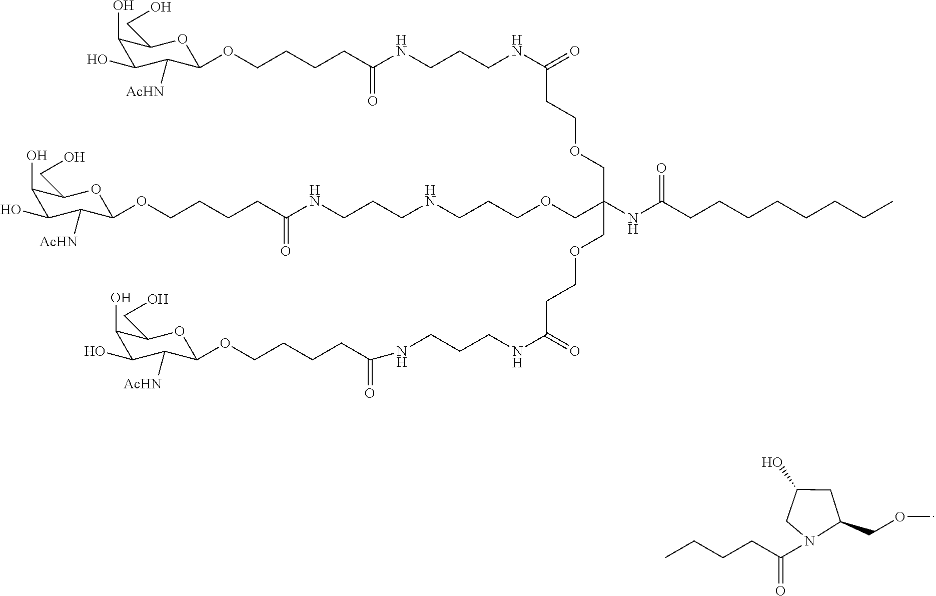

As used herein, the terms 2′-deoxy-2′-fluoroadenosine and 2′-fluoroadenosine may be used interchangeably.

As used herein, the terms 2′-deoxy-2′-fluorocytidine and 2′-fluorocytidine may be used interchangeably.

As used herein, the terms 2′-deoxy-2′-fluoroguanosine and 2′-fluoroguanosine may be used interchangeably.

As used herein, the terms 2′-deoxy-2′-fluorouridine and 2′-fluorouridine may be used interchangeably.

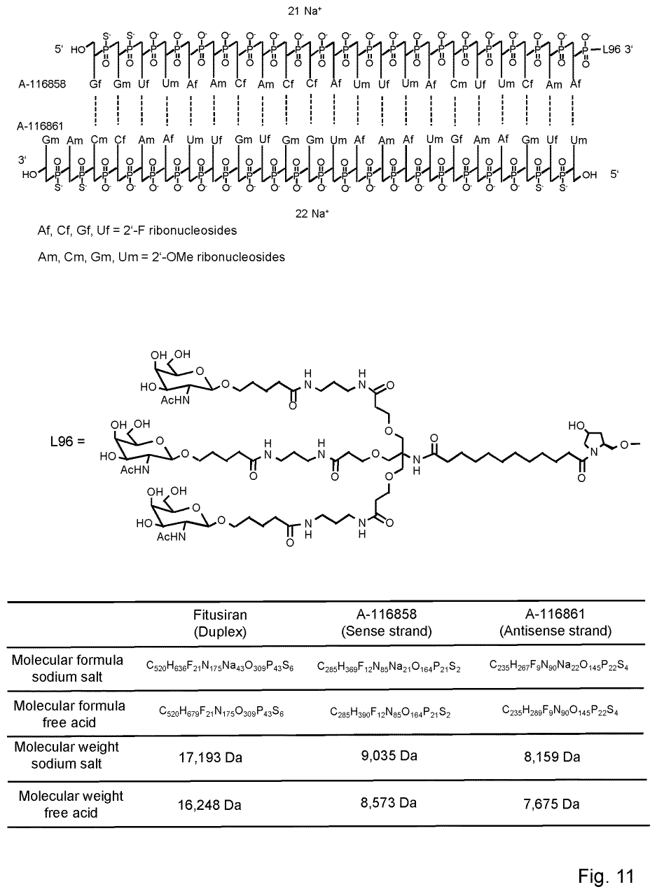

The expanded structural formula, molecular formula, and molecular weight of fitusiran are shown in FIG. 11.

The structure of fitusiran can also be described using the following diagram, wherein the X is O:

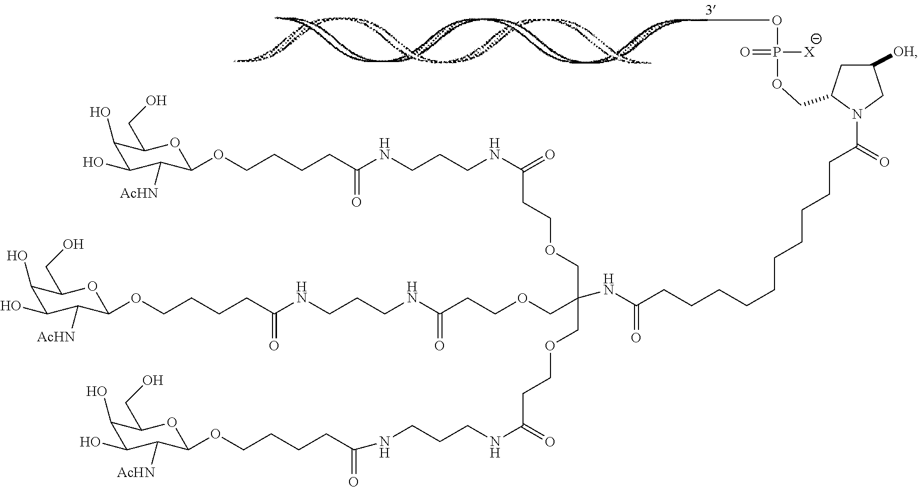

### Fitusiran is Shown in FIG. 11 in Sodium Salt Form.

In some embodiments, the device delivers fitusiran in an aqueous solution, wherein fitusiran is at a concentration of about 40 to about 200 mg/mL (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

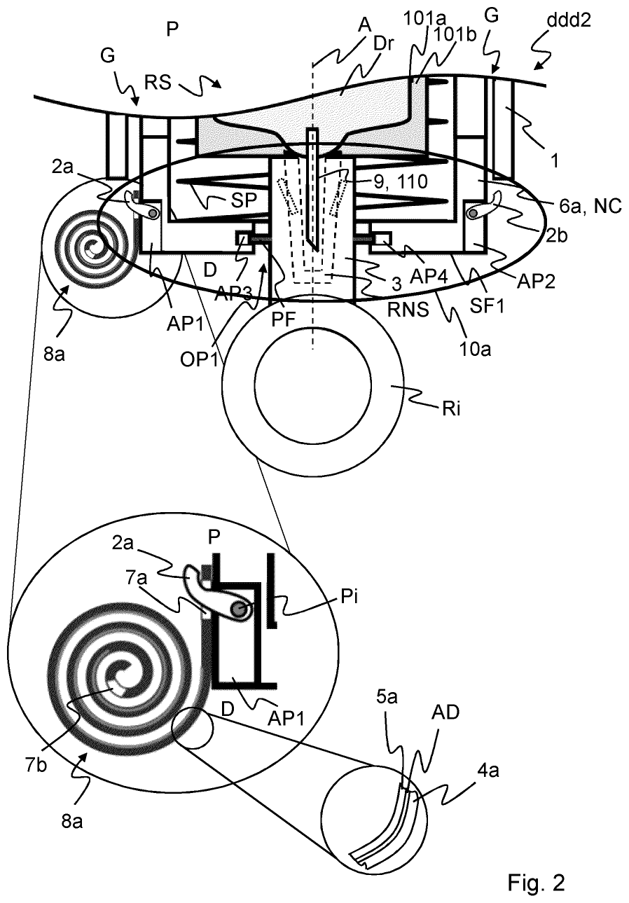

FIG. 2 illustrates an auto-injector ddd2 according to a second embodiment. The auto-injector ddd2 may comprise:

An essentially cylindrical housing 1,
A needle shield 6a, NC, e.g. a movable shield that is movable relative to housing 1 along longitudinal axis A from a first state (needle 9, 110 covered by needle shield 6a, NC) to a second state (needle 9, 110 exposed or not covered by needle shield 6a, NC),
A spring SP that biases needle shield 6a distally D from housing 1,
A drug container 101a comprising a drug Dr,
A retaining member 101b for retaining the drug container 101a within a retaining space RS within housing 1, e.g. a separate container carrier that is a separate part from housing 1 or a retaining member 101b that is an integral part of housing 1, and/or
A cap 3, 112 that is described below in more detail.

Needle shield 6a, NC may comprise a distal surface SF1, BF that extends essentially perpendicular to longitudinal axis A and that is configured to be pressed against the skin SK of a patient Pat during usage of auto-injector ddd2. Surface SF1 may be rotatory symmetrical or essentially rotatory symmetrical, e.g. ring shaped as well as other parts of the auto-injector ddd2 illustrated in FIG. 2, e.g. housing 1, drug container 101a, etc. There may be a central opening OP1 within surface SF1. Central opening OP1 may allow passage of cap 3 and/or passage of needle 9, 110 during needle insertion and during drug Dr injection.

Needle shield 6a, NC may carry a piercing closure assistance member 10a. The piercing closure assistance member 10a may comprise at least two or exactly two hook features 2a, 2b (holding element(s)). The first hook feature 2a may be arranged within a first lateral aperture AP1 within needle shield 6a, NC. The second hook feature 2a may be arranged within a second lateral aperture AP2 within needle shield 6a, NC. Aperture AP1 may be arranged at an opposite side of needle shield 6a, NC compared to the location of aperture AP2.

The first hook feature 2a may be arranged pivotable around a pivot axis Pi that extends essentially in the circumferential or angular direction with respect to longitudinal axis A of drug delivery device ddd2. A first end portion of first hook feature 2a may have a hole for a pivot axis member or the pivot axis member may be formed integral with the first end portion of first hook feature 2a. Hook feature 2a may be an elongated part that extends from the first end portion to a middle portion e.g. comparably straight. There may be a second end portion that extends from the middle portion in a curved manner. Thus, there may be a concave aperture within the second end portion that is adapted to hold ends of the piercing closure element 8a as is described below in more detail. The second end portion of hook feature 2a may extend laterally from needle shield 6a, NC into the way of housing 1 since there may be only a small circumferential gap G between housing 1 and needle shield 6a, NC. Thus, the free second end portion of hook feature 2a may be pivoted by housing 1 around pivot axis Pi. The same may apply to hook feature 2b that may have e.g. the same shape and the same function as hook feature 2a.

The piercing closure element 8a (e.g. a bandage or a band-aid) may have the shape of an elongated stripe. The stripe may be rolled up or folded in some way. In another embodiment the stripe may be flat if no external forces are applied to it. Piercing closure element 8a may comprises a stack of layers, e.g. in the following sequence: s
A tape 4a (basis layer), e.g. a stretch or stretchable tape 4a, and/or
An adhesive layer AD, and/or
A cover foil 5a (cover layer) that may prevent hardening of the glue within adhesive layer AD.

Adhesive layer AD may optionally be integral part of tape 4a (basis layer).

Piercing closure element 8a may comprise two apertures 7a, 7b on opposite end portions as is described in more detail below, see FIG. 6. One end portion of piercing closure element 8a may already be attached to first hook feature 2a during assembling of auto-injector ddd2 in a factory. However, according to another embodiment, a user or patient Pat of auto-injector ddd2 may attach the first end portion of piercing closure element 8a to auto-injector ddd2 manually, e.g. by inserting the free end portion of hook feature 2a through aperture 7a of piercing closure element 8a thereby observing that cover foil 5a is at the outside.

According to an embodiment a comparably small diameter cap 3, 112 is used that has an outer diameter that is less than the outer diameter of needle shield 6a, NC. Cap 3 may comprise:
A main cylindrical portion,
A ring Ri portion that is appropriate to pull cap 3 from auto-injector ddd2 prior to usage, and
At least one optional lateral protruding feature PF.

The main cylindrical portion of cap 3 may comprise a rigid needle shield RNS (or a flexible needle shield). Rigid needle shield RNS may be hold within cap 3 by a cage feature or grabber, e.g. by a metallic cage or grabber. The cage/grabber may comprise barbs that are shaped as hooks or prongs, see dotted rectangles in FIG. 2.

The at least one lateral protruding feature PF may extend into lateral apertures AP3, AP4 arranged within an inner surface of opening OP1 of needle shield 6a, NC in order to hold cap 3, 112 in place. However, other holding principles may be used as well, e.g. press fit, screw thread, etc.

According to another embodiment cap 3 may have a larger outer diameter/width compared to the outer diameter of needle shield 6a, NC. Thus, piercing closure element 8a may be attached to hook features 2a, 2b only after removal of the large diameter/width cap.

Hook features 2a, 2b may be hold in the upper position (first state) by spring elements (not illustrated) and or by small protrusions, e.g. clamping features. The same elements may be used to hold hook features in the lower position (second state), e.g. spring element, small protrusions, etc. However, optionally hook features 2a, 2b may snap back to the first position if no external forces are applied.

It is of course possible to use other holding features than hook features 2a, 2b, e.g. ramped protrusions, round protrusions, flexible elements, etc.

According to another embodiment, a purely piercing closure element application device may not comprise parts for drug delivery and/or a needle 9, 110. Thus, hook features 2a, 2b or other holding elements may be held by a part that comprises contact surface SF1 but that is not a movable needle shield 6a.

Thus, the drug delivery device 100, ddd2 may comprise:
A main housing body 1, and
Preferably a retaining space RS for the drug container 101a within the main housing body 1, and
A movable shield 6a, NC that is axially movable relative to the main housing body 1.

The movable shield 6a, NC may be configured to cover the drug administration member 9, 110 at least in a first state and to expose the drug administration member 9, 110 in a second state. The movable shield 6a, NC may comprise the piercing closure assistance member 10a.

The piercing closure assistance member 10a may comprise at least one holding element that is configured to hold the piercing closure element 8a. The at least one holding element may be configured and/or arranged such that the relative movement between the movable shield 6a, NC and the main housing body 1 releases the piercing closure element 8a from the at least one holding element, preferably when the movable shield 6a, NC moves from the first state to the second state.

Preferably, the piercing closure element 8a may extend from a first holding element of the at least one holding elements over a contact surface SF1 of the movable shield 6a, NC to a second holding element of the at least one holding element.

FIG. 3 illustrates the auto-injector ddd2 wherein a user or patient Pat has pulled off cap 3, e.g. using ring Ri. Thereafter, the user has pulled piercing closure element stripe 8a from hook feature 2a to hook feature 2b. Aperture 7b of piercing closure element stripe 8a has been hanged up over hook feature 2b, e.g. within concave portion of hook feature 2a. Thus, a portion of piercing closure element 8a stripe may be arranged in front of distal opening OP1 of a movable needle shield 6a, NC. Moreover, piercing closure element 8a may be in a stretched state, see stretching As as indicated by arrows in FIG. 3. In the stretched state piercing closure element 8a may comprise a central portion CP that has the same thickness as the end portions of piercing closure element 8a or that is thinner than the end portions of piercing closure element 8a. However, stretching may be optional as well as usage of a thinner portion.

As is illustrated in FIG. 3, cover foil 5a is not attached to piercing closure element 8a any more since the user or patient Pat has removed it, e.g. using tabs as illustrated in FIG. 6.

FIG. 3 illustrates an axial distance Di1 between distal surface SF1, BF and a distal end of housing 1. Distance Di1 is the distance when needle shield 6a, NC is not yet pushed against the skin SK of patient Pat, e.g. needle shield 6a, NC is in a first state in which needle 9, 110 is covered and protected by needle shield 6a, NC. In the first state of needle shield 6a, NC, pivot axis Pi of hook features 2a and 2b is arranged below or distal of distal end of housing 1. Therefore, hook features 2a and 2b are still in their initial position, e.g. the second end portions are directed proximally or upwards.

FIG. 4 illustrates the auto-injector ddd2 in a state in which needle 9, 110 is inserted and drug Dr delivery may commence. Thus, needle shield 6a, NC is in its second state in which needle 9, 110 is exposed, e.g. outside of needle shield 6a, NC. Thus, needle 9, 110 pierces into the skin SK of patient Pat in order to form a puncture Pu within skin SK. Spring SP may be in a compressed state and there is an axial distance Di2 that is less than distance Di1, e.g. less than half of distance Di1. Moreover, pivot axis Pi of hook features 2a and 2b is arranged proximally of distal ends of housing 1. Therefore, free ends of hook features 2a and 2b are directed downwardly, e.g. in a second state in which they release piercing closure element 8a. Release of piercing closure element 8a may be promoted further by pushing the ends of piercing closure element 8a away from hook features 2a and 2b by the distal end of housing 1.

Drug Dr may be injected through needle 9, 110 into the skin SK of patient Pat. After injection, auto-injector ddd2 may be removed from the skin SK of patient Pat.

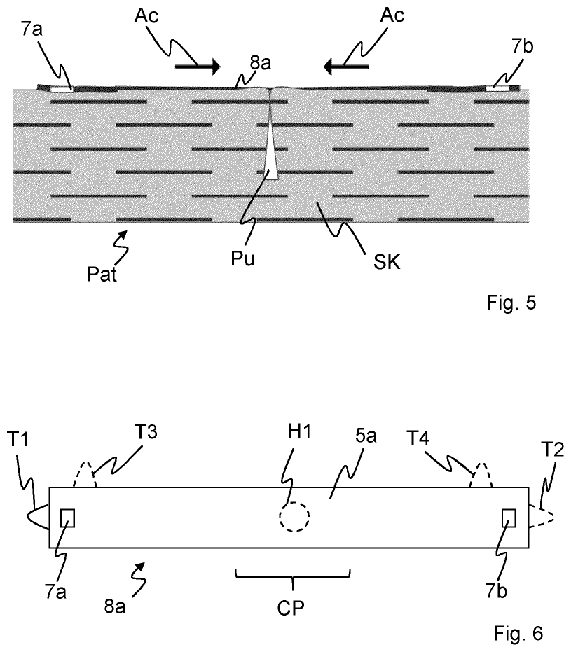

FIG. 5 illustrates the skin SK of patient Pat after injection of the drug and with piercing closure element 8a stripe left on skin SK in a contracted conformation, see contraction Ac as indicated by two arrows. As is illustrated in FIG. 5, an opening of puncture Pu is closed by the contraction Ac of piercing closure element 8a. Thus, wound healing and/or sterility may be promoted, e.g. independent of the fact that the opening of puncture Pu is covered by piercing closure element 8a (no hole H1) or not (hole H1) as is described in the following.

FIG. 6 illustrates the piercing closure element 8a stripe used in the second embodiment in a top view. A longitudinal length of piercing closure element 8a may be greater than the width, e.g. a maximum width of piercing closure element 8a, e.g. in a direction that is perpendicular to a longitudinal axis of piercing closure element 8a. The length may be more than twice, more than three times the width or more than for times, etc. The length may be less than 20 times the width or less than 10 times the width of piercing closure element 8a.

There may be an optional hole H1 within a central portion CP of piercing closure element 8a. Hole H1 may allow passage of needle 9, 110 during needle insertion. Hole H1 may have a circular, elliptical or other shape.

In another embodiment no separate hole H1 is used and needle 9, 110 may be pierced through the remaining layers of piercing closure element 8a during needle insertion and/or drug Dr injection.

At least one lateral tab T1 to T4 of cover layer 5a may ease removal of cover foil 5a, preferably after piercing closure element 8a has been attached to both hook features 2a and 2b. The at least one tab T1 to T4 may extend laterally from tape layer 4a and/or adhesive layer AD. In another embodiment there are regions in which no adhesive is applied to tape layer 4a in order to ease removal of cover foil or cover layer 5a.

Apertures 7a, and 7b may have a rectangular, quadratic, circular, elliptical or other shape. The locations of apertures 7a and 7b may be modified as well. The edges of piercing closure element 8a may be sharp, e.g. having an angle of about 90 degree, e.g. in order to ease release of piercing closure element 8y from hook features 2a and 2b or from other appropriate holding features. However, rounded corners may be used as well.

A method may be provided for preparing a piercing closure assistance member 10a, comprising:
Applying a piercing closure element 8a to the piercing closure assistance member 10a according to any one of the embodiments mentioned above or to the piercing closure assistance member 10a of a drug delivery device 100, ddd2 according to one of the embodiments mentioned above.

The method may preferably comprise:
Fastening at least one end of the piercing closure element 8a, e.g. a band-aid, preferably manually before applying the piercing closure element 8a to the skin SK of a patient Pat and/or before injecting a drug Dr into the body of the patient Pat by using the drug delivery device 100, ddd2, and/or
Preferably, putting a lateral aperture 7a, 7b of the piercing closure element 8a over a holding feature 2a, 2b of/on the drug delivery device 100, ddd2 or of another piercing closure element application device.

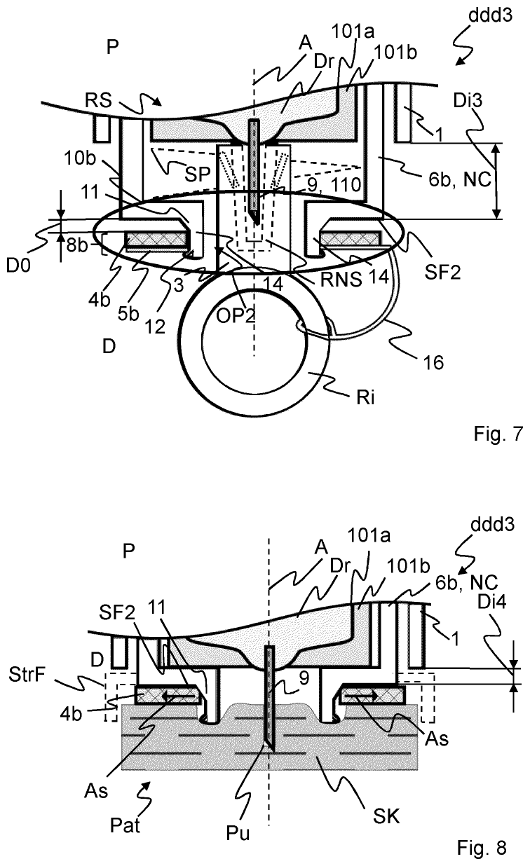

FIG. 7 illustrates an auto-injector ddd3 according to a third embodiment comprising e.g. a ring shaped piercing closure element 8b arranged around a distal opening OP2 of a movable needle shield 6b, NC. Auto-injector ddd3 may comprise:
An essentially cylindrical housing 1,
A needle shield 6b, NC, e.g. a movable shield that is movable relative to housing 1 along longitudinal axis A from a first state (needle 9, 110 covered by needle shield 6b, NC) to a second state (needle 9, 110 exposed or not covered by needle shield 6b, NC),
An optional spring SP that biases needle shield 6a distally D from housing 1, e.g. there may be no spring SP, e.g. because there is an axially slidable medicament container/syringe or because another spring is used to bias needle shield 6b, NC distally,
A drug container 101a comprising a drug Dr,
A retaining member 101b for retaining the drug container 101a within a retaining space RS within housing 1, e.g. a separate container carrier that is a separate part from housing 1 or a retaining member 101b that is an integral part of housing 1, and/or
A cap 3, 112 that is described in more detail below.

Needle shield 6b, NC may comprise a distal surface SF2 that extends essentially perpendicular to longitudinal axis A of auto-injector ddd3 or of another drug delivery device. Needle shield 6b, NC may be configured to be pressed against the skin SK of a patient Pat during usage of auto-injector ddd3. Surface SF2 may be rotatory symmetrical or essentially rotatory symmetrical, e.g. ring shaped as well as other parts of the auto-injector ddd3 illustrated in FIG. 7, e.g. housing 1, drug container 101a, etc. There may be a central opening OP2 within surface SF2. Central opening OP2 may allow passage of cap 3 and/or passage of needle 9, 110 during needle insertion and/or during drug Dr injection.

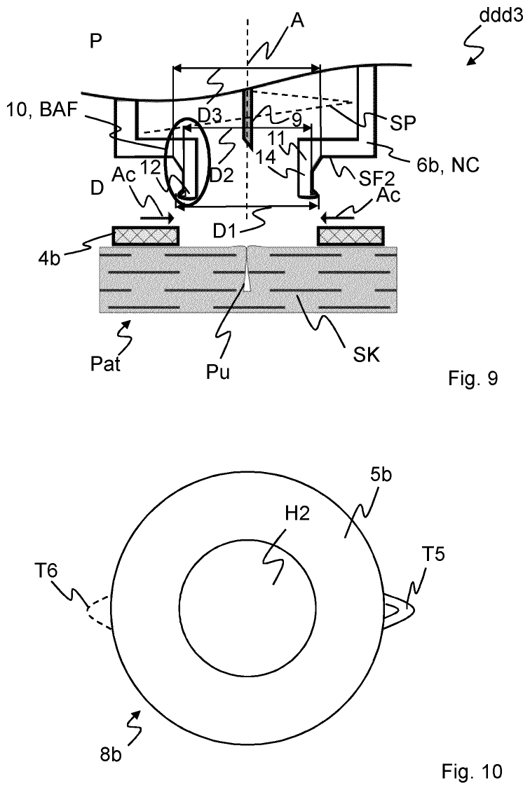

Needle shield 6b, NC may carry a piercing closure assistance member 10b. The piercing closure assistance member 10b may comprise in the following sequence form the proximal end to the distal end:
A contact surface SF2, e.g. a contact surface that extends essentially radially outwards,
A chamfer or ramp portion 11, e.g. a stretching portion that has an outer maximum width or outer maximum diameter D3, see FIG. 9 that may smaller than the outer diameter or maximal outer width of the contact surface SF2,
A constant diameter or maximal width middle portion, e.g. having the same outer diameter D2 along its axial extension, see FIG. 9 or the same maximal outer width as at the beginning of the stretching portion, e.g. a cylindrical portion 14, and/or
A retaining (retainer) portion 12 having a larger outer dimeter/width D1, see FIG. 9 than the middle portion, e.g. a circumferential flange or at least one flange portion.

According to another embodiment, ramped portion 11 (chamfer) is omitted and piercing closure element 8b is applied in a non-stressed conformation or in an essentially non-stressed conformation.

The piercing closure element 8b (e.g. a bandage or a band-aid) may have the shape of a ring or it may have a quadratic or other outer shape comprising a central hole. Piercing closure element 8b may comprises a stack of layers, e.g. in the following sequence:
A tape 4b (basis layer), e.g. a stretch or stretchable tape 4b, and/or
An adhesive layer AD, and/or
A cover foil 5b (cover layer) that may prevent hardening of the glue within adhesive layer AD.

Adhesive layer AD may optionally be integral part of tape 4b (basis layer).

Piercing closure element 8b may comprise a central opening through which piercing closure assistance member 10b may protrude. Piercing closure element 8b is described in more detail below, see FIG. 10 and corresponding description.

According to an embodiment a comparably small diameter cap 3 is used that has an outer diameter that is less than the outer diameter of needle shield 6a, NC. Cap 3 may comprise:
A main cylindrical portion, and
A ring Ri portion that is appropriate to pull cap 3 from auto-injector ddd2 prior to usage.

The main cylindrical portion of cap 3 may comprise a rigid needle shield RNS (or a flexible needle shield). Rigid needle shield RNS may be hold within cap 3 by a cage feature or grabber, e.g. by a metallic cage or grabber. The cage/grabber may comprise barbs that are shape as hooks or prongs, see dotted rectangles in FIG. 7. Cap 3 may be hold in place by using e.g. press fit, screw thread, etc.

A filament (wire) portion 16 may be used to connect cover foil 5b with ring Ri, e.g. in order to couple removal of cap from auto-injector ddd3 or other drug delivery device with removal of cover foil 5b from tape layer 4b.

According to another embodiment, a cap similar to cap 3 may have a larger outer diameter/width compared to the outer diameter of needle shield 6b, NC. Thus, piercing closure element 8b may be covered by the cap. A cover foil 5b may be used optionally thereby. No filament (wire) portion 16 may be necessary thereby. However, if a cover foil 5b is used together with the broader cap, a filament (wire) portion 16 may be used as well. Filament (wire) portion 16 may be fastened to the cap at the inside of the cap or at the outer side thereof.

There may be a distance DO between a proximal side of piercing closure element 8b and surface SF2 in an initial state of auto-injector ddd3 or of another drug delivery device. Distance DO may correspond to the axial length of ramped portion 11 (chamfer). Thus, piercing closure element 8b may not be stretched or may only be pre-stressed to a minor stress level in the initial state, e.g. compared with the lateral stress during application of piercing closure element 8b to the skin SK of a patient.

Moreover, in the initial state there may be an axial distance Di3 between surface SF2 and a distal end of housing 1.

In another embodiment, piercing closure assistance member 10b may be arranged on a drug delivery device that is not an auto-injector, e.g. on a pen-type device of the extendable length type as described above. Piercing closure assistance member 10b may be arranged on a connection element that is used to couple a needle to a cartridge, e.g. to a cartridge comprising a ferrule and/or a distal closure (e.g. a septum) that has to be pierced by a needle.

In another embodiment, a purely piercing closure element application device may not comprise parts for drug delivery and/or a needle 9, 110. Thus, piercing closure assistance member 10 may not be arranged on a movable needle shield.

Thus, a drug delivery device 100, ddd3 may comprise piercing closure assistance member 10b, BAF. Piercing closure assistance member 10b, BAF may comprise at least one holding feature that may configured to extend through a central opening of the piercing closure element 8b and to hold the piercing closure element 8b on the holding feature. Preferably, the holding feature may comprise a retaining feature 11 that may extend radially outwardly.

In use, a user/patient Pat removes cap 3 from auto-injector ddd3 or another drug delivery device. Thereby, also cover foil 5b is removed from tape layer 4b.

FIG. 8 illustrates the auto-injector ddd3 according to the third embodiment in a state in which the needle 9, 110 is inserted into the skin SK of a patient Pat and drug delivery may commence. There is an axial distance Di4 between surface SF2 and the distal end of housing 1. Distance Di4 may be much smaller than distance Di3 due to a relative movement between housing 1 and needle shield 6b, NC, e.g. less than half of distance Di3. Contrary to the second embodiment, this relative movement is not used to release piercing closure element 8b.

Piercing closure element 8b is released in the following way. Pushing of auto-injector ddd3 or other drug delivery device against skin SK pushes piercing closure element 8b proximally against surface SF2 and along of ramped portion 11 (chamfer). Ramped portion 11 (chamfer) widens the central hole H2, see FIG. 10 of piercing closure element 8b and/or stretches piercing closure element 8b laterally. At the same time, skin SK presses against the distal D side of piercing closure element 8b thereby making sticky contact to adhesive layer AD.

Again, needle 9, 110 generates a puncture Pu within the skin SK of patient Pat. The distal rim of needle shield 6b, NC may extend into the skin SK of patient Pat. This may have positive effects for needle 9, 110 insertion and/or drug delivery. Optional stretching As of piercing closure element 8b, e.g. a band-aid is indicated by arrows in FIG. 8. Stretching the piercing closure element could also stretch the needle insertion site of the skin to reduce pain perception and/or to ensure a better defined needle insertion and/or could help to avoid the back flow of the drug out of the needle insertion site after removal of the device from the skin, see, e.g., FIG. 9.

According to a further embodiment, skin SK may be stretched by stretching features StrF on drug delivery device ddd3 or on another device used to apply piercing closure member 8b, e.g. the remaining tape layer 4b thereof. This may be in addition to stretching of piercing closure member 8b or tape layer 4b by ramped portion 11 or instead of usage of ramped portion 11. According to a further embodiment, stretching features StrF may be more rounded features. Stretching features StrF may be circumferential features that extend along the whole circumference of needle shield 6b, NC. According to a further embodiment, stretching features StrF may not extend along the whole circumference of needle shield 6b, NC but may be formed as arms, e.g. as resilient arms that may be arranged on opposite lateral sides of needle shield 6b, NC or of contact surface SF2.

FIG. 9 illustrates auto-injector ddd3 according to the third embodiment after removal of auto-injector ddd3 or other drug delivery device from skin SK of patient Pat. After injection of drug Dr, piercing closure element 8b may be left on skin SK in a contracted conformation, see contraction Ac as indicated by arrows. As is illustrated in FIG. 9, an opening of puncture Pu is closed by the contraction Ac of piercing closure element 8b. Thus, wound healing and/or sterility may be promoted, independent of the fact that piercing closure element 8b does not cover the opening of puncture Pu since hole H2 is used as described in the following.

Further, FIG. 9 illustrates the following diameters/maximal widths D1 to D3 that are also mentioned above:
Outer diameter/width D1 of distal flange 12,
Outer diameter/width D2 of cylindrical portion 14, and
Outer diameter/width D3 of ramped portion 11 on surface SF2.

Outer diameter/width D3 may be larger than outer diameter/width D1 and/or outer diameter/width D2. Outer diameter/width D1 may be larger than outer diameter/width D2.

If outer diameter/width D1 is less than diameter/width D3, release of piercing closure element 8b from piercing closure assistance member 10 may be promoted since the hole H2 of piercing closure element 8b is widened to a diameter that is greater than the outer diameter D1 of the distal flange. Thus, the distal flange can be removed through the widened hole H2 easier, e.g. if there is a relaxation time of tape layer 4b and/or skin SK against immediate contraction of piercing closure element 8b.

Contraction Ac of piercing closure element 8b or of tape layer 4b is illustrated by arrows in FIG. 9. However, stretching and contraction may be optional as mentioned already above several times.

FIG. 10 illustrates the ring shaped piercing closure element 8b used in the third embodiment in a top view. According to the embodiment, piercing closure element 8b may have a circular outer shape. In other embodiments, piercing closure element 8b may have an elliptical, oval or other outer shape. There may be a hole H2 within a central portion of piercing closure element 8b. Hole H2 may allow passage of needle 9, 110 during needle insertion. Hole H2 may have a circular, elliptical or other shape.

At least one lateral tab T5 and/or T6 of cover layer 5b may ease removal of cover foil 5b, preferably after piercing closure element 8b has been attached to piercing closure assistance member 10. The at least one tab T5 and/or T6 may extend laterally from tape layer 4b and/or adhesive layer AD. In another embodiment, there are regions in which no adhesive is applied to tape layer 4b in order to ease removal of cover foil or cover layer 5b. The at least one lateral tab T5 and/or T6 may have an opening that allows to put filament 16 through in order to connect cover foil 5b and cap 3.

A method for preparing a piercing closure assistance member 10b, may comprise:
Applying a piercing closure element 8b to the piercing closure assistance member 10b according to any one of the embodiments mentioned above or to the piercing closure assistance member 10b of a drug delivery device 100, ddd3 according to one of the embodiments mentioned above.

The method may preferably comprise:
Attaching the piercing closure element 8b, e.g. a band-aid preferably automatically to drug delivery device 100, ddd3 or to another piercing closure element application device by arranging a central opening H2 of the piercing closure element 8b over piercing closure assistance member 10b of drug delivery device 100, ddd3 or of another device.

Thus, the concepts may be applied to an auto-injector with fixture on skin/skin preparation. Skin preparation could be cleaning and/or squeezing and/or stretching prior to the injection. This document describes the use of e.g. elastic bandages/piercing closure elements 8a, 8b e.g. in order to reduce backflow after an injection with an auto-injector ddd2, ddd3. The document describes the design of features for an auto-injector, e.g. ddd2, ddd3 that may allow the injector to apply the bandage 8a, 8b while it is used. The auto-injector ddd2, ddd3 may not be entirely described in this document as it is assumed that it may work (aside from described new features) similar to known drug delivery devices.

The bandage carrier material, e.g. 4a, 4b may be made from a flexible material that may allow it to be applied with a significant stretch, that may enable it to pull together the skin SK at puncture Pu after the injection has occurred. For example, bandage 8a, 8b could use material similar to a kinesio-tape, e.g. cotton with elastane or highly elastic artificial silk. Other options would be rubberlike materials. However, stretching may be optional. Thus, piercing closure elements or bandages 8a, 8b may be applied that are not stretched or that are only stretched to a minor degree.

Certain glue pattern, e.g., in wavy way on the bandage/band aid could support efficient stretching and relaxation after injection to suppress, e.g., the backflow of drug.

In a first variant, the bandage may be similar to a small piece of kinesio-tape. After removing cap 3, the user/patient Pat may apply the bandage by hooking it in at one side of the needle shield, e.g. 6a, NC and stretching it until it can be fixated at another position on the needle shield, e.g. 6a, NC. In this variant the bandage can be a stripe or rolled up before use. As mentioned above, stretching may be optional. Thus, hooks or hook features 2a, 2b may be part of a piercing closure assisting member 10a that may also be named more specifically as bandage application feature (BAF) 10a.

In a second variant, a pre-attached or simple to attach, flexible, e.g. ring formed bandage, e.g. 8b that may be widened by a piercing closure assisting member 10b or bandage application feature (BAF) 10b on the device, e.g. ddd3 may be used. The aim of this feature may be to first spread the bandage/piercing closure element, e.g. 8b before applying the necessary pressure to glue it on the user's skin SK. The BAF depicted does this via a spreading chamfer which may widen the bandage (piercing closure element), e.g. 8b slightly before applying it.

Other examples for a BAF may be:
Solutions that use arms of the needle shield that spread due to features on the main housing body that deflect them.
Springs or resilient features that are released when removing the cap, e.g. a cap 3, 112 to widen the bandage.
Electromechanical actuators that may widen the bandage/piercing closure element.

In a third variant, a bandage may be used that is not stretched in advance but applies stress by deformation after being applied, e.g. by including a shape memory alloy in the bandage carrier material.

In a fourth variant, a bandage/piercing closure element is used that is not-stressed at all or that is only marginally stressed. This bandage may cover the puncture Pu or may not cover puncture Pu.

The following may apply to the elastic bandage, e.g. 8a, 8b:
May apply stress on the skin that closes the wound. Preventing backflow and improving wound healing.
Variant 1: Similar to a kinesio-tape,
Variant 2: Rubberlike carrier material,
May have one or more of the following features:
   Hole H1, H2 that the needle 9, 110 can pierce through.
   Lugs, Latches or other features (apertures 7a, 7b) to connect it to the needle shield 6a, 6b, NC,
   Shape memory alloy to may provide stress, e.g. due to higher temperature of skin SK.

The following may apply to bandage application feature BAF, 10a, 10b:
Broadest term for a feature that is part of the device and that has the purpose to apply, e.g. to spread a bandage/piercing closure element, e.g. 8a, 8b prior to or while the injection takes place and/or applying the bandage/piercing closure element, e.g. 8a, 8b to the skin SK.
Examples for solutions:
   Spreading chamfer or another element, e.g. a ramped portion 11,
   Flexible arms,
   Springs and/or resilient elements,
   Actuators, e.g. electromechanical actuators.

The concept to use a stretched bandage/piercing closure element, e.g. 8a, 8b or a non-stretched bandage to close an injection site might also be implemented on manual injection devices. In cases where the device is repeatedly used, the bandages/piercing closure elements could be exchangeable and/or replaceable single use parts. Also devices that only apply a stretched bandage/piercing closure element prior to an injection of any sort would be possible, e.g. the device for applying the stretched bandage or the non-stretched bandage may be a separate device with regard to an injection device.

With reference to the embodiment illustrated in FIGS. 2 to 6 the following use process may be applied: The user may remove cap 3 from the device and may hook one aperture (lug) 7a of the bandage 8a (piercing closure element) over a hook feature 2a of the device ddd2. Then he/she may stretch bandage 8a over the bottom side of needle shield 6a, NC and he/she may secure it by attaching another aperture (lug) 7b into another hook feature 2a. The cover foil 5a protecting the bandages glue may be removed manually. When the user pushes the autoinjector ddd2 on his skin SK the glue may fixate the bandage 8a on the skin SK in a stretched position. Pushing further, needle shield 6a may move into housing 1. While doing so, hook features 2a and 2b may get bent aside releasing apertures (lugs) 7a and 7b of bandage 8a.

During injection, needle 9, 110 may either pierce through stretched bandage/piercing closure element 8a or bandage/piercing closure element 8a may have a specific injection hole H1 which may allow needle 9 to pass through. After needle 9 is removed the stretched bandage 8a may pull skin SK together around puncture Pu, which may prevent bleeding and may improve wound healing, especially when e.g. an FLAI is used, i.e. a fast and large autoinjector.

With reference to the embodiment illustrated in FIGS. 7 to 10 the following may be valid. In order to apply a bandage/piercing closure element that may seal the injection site, a bandage (piercing closure element) application feature (BAF) 10b may be attached to or may be included into needle shield 6b, NC. BAF 10b may allow to administer a stretchable bandage with a hole H2 in a way that pinches the skin SK together after injection.

BAF 10a, 10b is mentioned in the description above and in the claims as “piercing closure assisting member”, e.g. as it is used to assist in closing a piercing/puncture Pu using a piercing closure element 8a/8b.

The BAF may consists of or may comprise a retainer 12 that prevents the bandage/piercing closure element from falling off and a spreading chamfer 11. Moreover, BAF 10 may comprise an optional cylindrical portion 14 arranged between retainer 12 and chamfer 11. During injection chamfer 11 may widen the elastic bandage 8b. Meanwhile, needle shield 6b, NC may push bandage/band-aid or piercing closure member 8b onto the skin SK allowing the glue to secure it preferably in a stretched position. After injection, the bandage 8b may pull the skin SK together in order to regain its initial form, helping to close the injection site without actually covering it. In this concept bandage 8b may be pre-assembled to autoinjector ddd3 and cover foil 5b of bandage 8b could be removed together with cap 3.

Spoken with other words, a piercing closure assistance member 10a, 10b for applying a piercing closure element 8a, 8b, may comprise:
A member holder 2a, 2b; 11, 12, 14 that is configured to hold the piercing closure element 8a, 8b while the piercing closure element 8a, 8b is applied to a piercing site on the skin SK of a subject Pat.

The skin SK may comprise a piercing puncture (Pu). The piercing closure member 8a, 8b may comprise a surface that may be configured to be adhesively attached to the skin SK of the subject Pat.

Furthermore, a drug delivery device 100, ddd2, ddd3 is provided, comprising:
A piercing closure assistance member 10a, 10b according to any one of the embodiments mentioned above.

A method for preparing a piercing closure assistance member 10a, 10b may comprise:
Applying a piercing closure element 8a, 8b to the piercing closure assistance member 10a, 10b according to any one of the embodiments mentioned above or to the piercing closure assistance member 10a, 10b of a drug delivery device 100, ddd2, ddd3 according to any one of the embodiments mentioned above.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 11.

1-15. (canceled)

16. A piercing closure assistance member for applying a piercing closure element, the piercing closure assistance member comprising:
a member holder configured to hold the piercing closure element while the piercing closure element is applied to a piercing site on the skin of a subject, and
an adhesive surface that is configured to be adhesively attached to the skin of the subject.

17. The piercing closure assistance member according to claim 16, wherein the piercing closure assistance member is configured to apply the piercing closure element in a stretched conformation.

18. The piercing closure assistance member according to claim 16, wherein the piercing closure assistance member is configured to stretch the skin before the adhesive surface of the piercing closure assistance member contacts the skin.

19. The piercing closure assistance member according to claim 16, further comprising:
a housing body,
a shield that is movably arranged relative to the housing body, and
at least one holding element arranged on the shield,
wherein the at least one holding element is configured to hold the piercing closure element, and
wherein the at least one holding element is further configured such that the relative movement between the shield and the housing body releases the piercing closure element from the at least one holding element.

20. The piercing closure assistance member according to claim 19, wherein the at least one holding element comprises a pivotable element,
wherein the relative movement of the shield and the housing body pivots the pivotable element from a first state to a second state,
wherein, in the first state of the pivotable element, the piercing closure element is held by the pivotable element, and
wherein, in the second state of the pivotable element, the piercing closure element is released or is releasable from the pivotable element.

21. The piercing closure assistance member according to claim 16, further comprising at least one holding feature that is configured to extend through a central opening of the piercing closure element and to hold the piercing closure element on the holding feature.

22. The drug delivery device, comprising a piercing closure assistance member for applying a piercing closure element, the piercing closure assistance member comprising:
a member holder configured to hold the piercing closure element while the piercing closure element is applied to a piercing site on the skin of a subject, and
an adhesive surface that is configured to be adhesively attached to the skin of the subject.

23. The drug delivery device according to claim 22, further comprising:
a main housing body,
a retaining space for a drug container defined within the main housing body,
a drug administration member, and
a movable shield that is axially movable relative to the main housing body,
wherein the movable shield is configured to cover the drug administration member at least in a first state and to expose the drug administration member in a second state, and
wherein the movable shield comprises the piercing closure assistance member, the piercing closure assistance member further comprising at least one holding element configured to hold the piercing closure element, wherein the at least one holding element is further configured such that the relative movement between the movable shield and the main housing body releases the piercing closure element from the at least one holding element, when the movable shield moves from the first state to the second state.

24. The drug delivery device according to claim 23, wherein the at least one holding element comprises a pivotable element,
wherein the relative movement of the movable shield and the main housing body pivots the pivotable element from a first state to a second state,
wherein, in the first state of the pivotable element, the piercing closure element is held by the pivotable element, and
wherein, in the second state of the pivotable element, the piercing closure element is released or is releasable from the pivotable element.

25. The drug delivery device according to claim 23, wherein the at least one holding element comprises a protrusion that extends radially outward from the movable shield,
wherein the protrusion is configured such that the relative movement of the movable shield and the main housing body pushes the piercing closure element over the protrusion, thereby releasing the piercing closure element from the protrusion.

26. The drug delivery device according to claim 22, wherein the piercing closure assistance member further comprises at least one holding feature configured to extend through a central opening of the piercing closure element and to hold the piercing closure element on the holding feature,
wherein the holding feature comprises a retaining feature that extends radially outwardly.

27. The drug delivery device according to claim 26, further comprising a drug administration member and a movable shield configured to cover the drug administration member at least in a first state and to expose the drug administration member in a second state,
wherein the movable shield comprises the at least one holding element configured to hold the piercing closure element, and
wherein the piercing closure assistance member is an integral part of the movable shield.

28. The drug delivery device according to claim 27, wherein the piercing closure assisting member is connectable to a distal end of the drug delivery device after assembling of the drug delivery device, in order to attach the drug administration element to the drug container,
wherein the drug delivery device further comprises a first connection feature that is configured to be connected to a second connection feature on the piercing closure assisting member.

29. The drug delivery device according to claim 26, wherein the piercing closure assisting member further comprises at least one stretching element,
wherein the stretching element is configured to stretch the piercing closure element when the piercing closure element is pushed against the skin of a patient and/or against the stretching element.

30. A method for preparing a piercing closure assistance member comprising a member holder configured to hold the piercing closure element while the piercing closure element is applied to a piercing site on the skin of a subject, and an adhesive surface that is configured to be adhesively attached to the skin of the subject, the method comprising:
applying a piercing closure element to the piercing closure assistance member.
